(19) United States
(12) Patent Application Publication
(10) Pub. No.: US 2014/0071259 A1
(43) Pub. Date: Mar. 13, 2014

(54) ECHO-VIDEO-IMAGE TRANSMISSION DEVICE AND VIDEO-IMAGE DISTRIBUTION SYSTEM

(71) Applicants: Takashi Odou, Tokyo (JP); Satoshi UNO, Tokyo (JP)

(72) Inventors: Takashi Odou, Tokyo (JP); Satoshi UNO, Tokyo (JP)

(73) Assignee: KYORITSU KIDEN CO., LTD., Tokyo (JP)

(21) Appl. No.: 14/020,221

(22) Filed: Sep. 6, 2013

(30) Foreign Application Priority Data
Sep. 8, 2012 (JP) 2012-197991

(51) Int. Cl. A61B 1/045

(52) U.S. Cl. CPC A61B 1/045
USPC 348/77

(57) ABSTRACT

Disclosed is an echo -video-image transmission device that uploads, onto a cloud server, an echo video image of a fetus of a prenatal patient that is output from an ultrasonic diagnostic device. The echo-video-image transmission device includes a transmission terminal unit and an IC card reader unit. When the IC card reader unit reads an IC card on which at least a patient identification code and a system identification code are recorded, the transmission terminal unit is triggered to start acquiring the echo video image that is output from the ultrasonic diagnostic device, and the transmission terminal unit uploads, onto the cloud server, the echo video image that has been acquired for a predetermined period of time, together with the patient identification code and a medical institution identification code that has been stored in the transmission terminal unit in advance.

The present application claims priority to Japanese Patent Application No. 2012-197991 filed on Sep. 8, 2012, which is herein incorporated by reference.

### BACKGROUND

1. Technical Field

The present invention relates to a system for distributing an echo video image of a fetus of a prenatal patient over the Internet.

2. Background Art

Conventionally, there have been services in medical institutions that provide a postnatal patient with a DVD containing a record of the fetus/baby and the patient before, during, and after childbirth. Such DVDs typically contain information such as echo video images of the fetus taken during prenatal checkups, videos of the baby after birth, video images of information useful after childbirth, and information/guidance about the medical institution.

In order to receive echo video images through such services, a prenatal patient first registers patient information by inputting, for example, the patient's name, expected due date, and email address through a touch-panel-type terminal provided in a medical institution, and the medical institution issues a magnetic card to the prenatal patient. Then, upon prenatal checkup, the staff of the medical institution uses the magnetic card to acquire echo video images from a client terminal and store the echo video images in a server set up in the medical institution. The stored echo video images are recorded on a DVD after childbirth, and the DVD is provided to the postnatal patient.

On the other hand, echo still images are created by taking out still images from the echo video images and are processed so that they can be browsed from external devices, and password-containing URL information for accessing the server in the medical institution is sent in an email to an email address registered in advance. In this way, echo still images can be browsed from mobile terminals etc. over the Internet, not only by the prenatal/postnatal patient herself but also by her family living far away.

In such services, however, only echo still images can be browsed over the Internet, and browsing of echo video images is not possible. In such services, echo video images have to be provided in DVDs, but creating such DVDs requires much time and effort of the medical institution's staff. Further, there are cases where, even if a DVD is provided to a prenatal/postnatal patient, the DVD cannot be replayed due to differences in standards depending on DVD playback devices.

Meanwhile, JP-A-2007-299408 discloses a method of uploading fetal echo video images recorded by a medical institution onto a server on the Internet and allowing the echo video images to be accessed and browsed from a personal computer.

With this method, the prenatal/postnatal patient's family etc. can view echo video images on-demand, over the Internet when the prenatal patient takes an ultrasonic examination at a medical institution. The specific steps are as follows. At the time of checkup at a medical institution, a prenatal patient brings a CD storing a serial number and basic contents supplied in advance from the medical institution to the prenatal patient. Upon ultrasonic examination, a doctor reads a barcode indicated on the CD with a barcode reader connected to a PC. When ultrasonic examination is started, the captured ultrasonic image is transmitted to a central server in the medical institution and is stored together with the serial number corresponding to the barcode that has been read. When the prenatal/postnatal patient inserts the CD received from the medical institution into a readout slot of a PC that can access the Internet, the PC can log into the central server of the medical institution automatically without manual operation, and the ultrasonic image of the prenatal patient corresponding to the serial number can be streamed or downloaded.

This method, however, essentially requires a CD containing a serial number and basic contents (i.e., software) for viewing ultrasonic images with a personal computer etc. Further, it is necessary to indicate a barcode corresponding to the serial number on the CD so that it can be read. Thus, upon acquiring the captured ultrasonic image into the PC, the medical institution's staff, such as a doctor, needs to read in the serial number indicated on the CD with a barcode reader and then separately input a signal for starting the acquisition of the ultrasonic image. Thus, there are demands from the staff of medical institutions that performs such examinations to minimize the trouble required at the time of acquiring ultrasonic images. Further, in this method, a prenatal/postnatal patient or her family cannot view the ultrasonic images unless the CD is at hand.

Furthermore, this method requires a central server connected to a PC for each medical institution. Thus, server maintenance, such as the updating of software on the server, must be done for each server. Thus, server maintenance requires time and effort of a server administrator, and also, each medical institution needs to incur the cost and space for setting up the server.

Thus, there has been a demand for a system that is more simple and easy to use for the prenatal/postnatal patient herself, her family, the staff of medical institutions, and server administrators in browsing fetal echo video images.

The present invention has been made in view of the aforementioned circumstances, and an objective thereof is to provide an echo-video-image transmission device that can complete the acquisition of an echo video image, without fail, in a medical institution simply by holding up an IC card once above a card reader (or touching a card reader with an IC card) at the time of acquisition, and that can thereby drastically reduce the time and effort of the staff of medical institutions in acquiring echo images.

Another objective of the present invention is to provide a video-image distribution system that, by setting up a server on the Internet in providing a service for browsing fetal echo video images, enables a prenatal/postnatal patient and/or her family to easily view fetal echo video images anywhere and anytime, and that can completely eliminate setup, management, and maintenance of servers conventionally performed in medical institutions and also the trouble associated with the browsing service, such as the creation of DVDs.

### SUMMARY

An aspect of the present invention for achieving the aforementioned objectives is an echo-video-image transmission device that uploads, onto a cloud server, an echo video image of a fetus of a prenatal patient that is output from an ultrasonic diagnostic device. The echo-video-image transmission device includes a transmission terminal unit and an IC card reader unit. When the IC card reader unit reads an IC card on which at least a patient identification code and a system identification code are recorded, the transmission terminal unit is triggered to start acquiring the echo video image that is output from the ultrasonic diagnostic device, and the transmission terminal unit uploads, onto the cloud server, the echo video image that has been acquired for a predetermined period of time, together with the patient identification code and a medical institution identification code that has been stored in the transmission terminal unit in advance.

### BRIEF DESCRIPTION OF THE DRAWINGS

FIG. 1 illustrates an overall configuration of an embodiment of the present invention.

FIG. 2 illustrates a configuration of a cloud server 60.

FIG. 3 illustrates a configuration of a reception terminal 40.

FIG. 4 illustrates a flowchart of an echo-video-image acquisition process.

FIG. 5 illustrates a flowchart of a transmission web service process.

FIG. 6 illustrates a flowchart of an echo-video-image conversion and storage process.

FIG. 7 illustrates a flowchart of a user browsing process.

### DETAILED DESCRIPTION OF EXEMPLARY EMBODIMENTS

At least the following matters will be made clear by the present specification and the accompanying drawings.

A first aspect of the present invention is an echo-video-image transmission device that uploads, onto a cloud server, an echo video image of a fetus of a prenatal patient that is output from an ultrasonic diagnostic device. The echo-video-image transmission device includes a transmission terminal unit and an IC card reader unit. When the IC card reader unit reads an IC card on which at least a patient identification code and a system identification code are recorded, the transmission terminal unit is triggered to start acquiring the echo video image that is output from the ultrasonic diagnostic device, and the transmission terminal unit uploads, onto the cloud server, the echo video image that has been acquired for a predetermined period of time, together with the patient identification code and a medical institution identification code that has been stored in the transmission terminal unit in advance.

In a second aspect of the present invention, when the IC card reader unit reads the IC card, the echo-video-image transmission device may determine whether the IC card is to be used in the echo-video-image transmission device on the basis of whether the medical institution identification code matches, and the echo-video-image transmission device may start to acquire the echo video image if the medical institution identification code matches, and may not acquire the echo video image if the medical institution identification code does not match.

In a third aspect of the present invention, the echo-video-image transmission device may use user registration information stored in the cloud server in advance as a basis for determining whether the IC card is to be used in the echo-video-image transmission device.

A fourth aspect of the present invention is a video-image distribution system including the aforementioned echo-video-image transmission device according to any one of the first to third aspects of the present invention, the aforementioned cloud server, and a reception terminal. The cloud server stores each echo video image uploaded from the echo-video-image transmission device in association with a medical institution identification code and a patient identification code. When receiving a request from the reception terminal to browse the echo video image, the cloud server performs user authentication, and then distributes, by streaming or downloading, the echo video image in a format compliant with the reception terminal.

In a fifth aspect of the present invention, the user authentication may be performed by using a password designated by a user at the tine of user registration performed in advance in association with the patient identification code and a security code described on the IC card.

According to the aforementioned aspects of the present invention, the acquisition of an echo video image can be completed, without fail, in a medical institution simply by holding up an IC card once above a card reader (or touching a card reader with an IC card) at the time of acquisition, and thereby the time and effort of the staff of medical institutions in acquiring echo images can be drastically reduced.

Further, because the server is set up in a centralized manner on a cloud over the Internet, in providing a service for browsing fetal echo video images, it is possible to enable a prenatal/postnatal patient and/or her family to easily view those echo video images anywhere and anytime, and completely eliminate setup, management, and maintenance of servers conventionally performed in medical institutions and also the trouble associated with the breaking service, such as the creation of DVDs.

### Embodiment of Present Invention

An embodiment of the present invention will be described below.

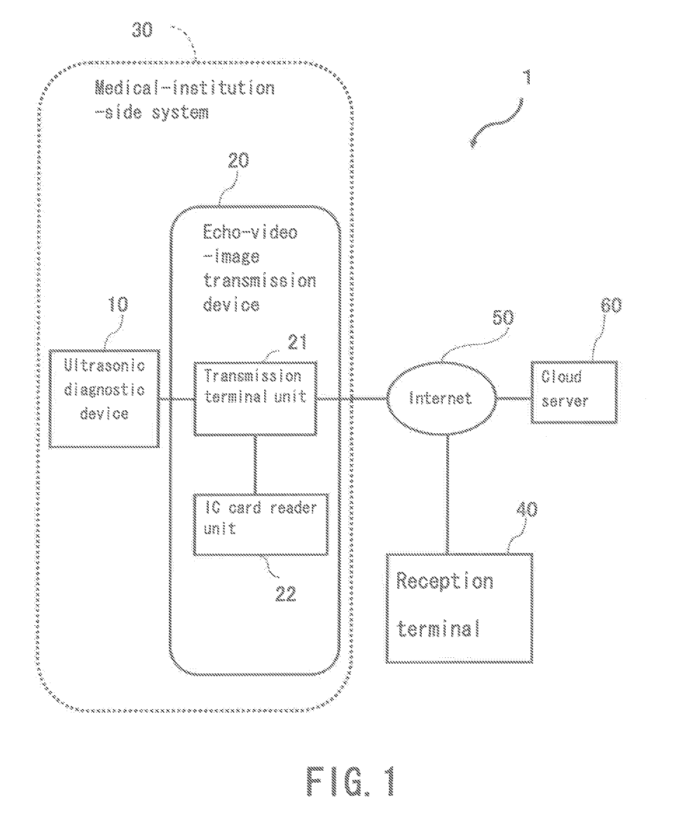

FIG. 1 illustrates the overall hardware configuration of an embodiment of the present invention. An echo-video-image transmission device 20 that acquires echo video images and transmits them to a server, a cloud server 60 consisting of a plurality of servers that receive, store, and manage the echo video images, and a reception terminal 40 for browsing the echo video images constitute a video-image distribution system, which is a server-client system, as a whole over the Internet.

Medical-Institution-Side Systems

As illustrated in FIG. 1, a medical-institution-side system 30 set up in each medical institution is made up of an echo-video-image transmission device 20, and an ultrasonic diagnostic device 10 that generates fetal echo video images. The echo-video-image transmission device 20 includes a laptop PC (personal computer) 21 as a transmission terminal unit 21 for acquiring echo video images generated by the ultrasonic diagnostic device 10 and uploading (transmitting) them onto the cloud server 60. A proximity-type IC card reader 22 capable of reading a proximity-type IC card is connected to the laptop PC 21. Here, a proximity-type card/reader compliant with ISO/IEC 14443 is used.

A capturing unit (not illustrated) is provided between the ultrasonic diagnostic device 10 and the transmission terminal unit 21 and allows video images or S-video from the ultrasonic diagnostic device 10 to be acquired from a USB terminal of the laptop PC 21. The laptop PC 21 is connected to the Internet and has an echo-video-image acquisition application program installed thereon. As described later, the laptop PC 21 acquires echo video images from the ultrasonic diagnostic device 10, and uploads them onto the cloud server 60 over the Internet for storage and browsing.

IC Card

An IC card is a card that can be read with the aforementioned proximity-type card reader, and is provided from a medical institution to a prenatal patient when, for example, the prenatal patient first visits the medical institution, in order to use the echo-video-image transmission device 20 and/or the video-image distribution system. The IC card provides a trigger (start signal) for starting the automatic acquisition of an echo video image, and information necessary for the prenatal/postnatal patient and her family to browse echo video images via the video-image distribution system is recorded/indicated on the IC card.

At the time of provision, the IC card records, in advance, at least: a patient identification code consisting of a character string of eight characters; and information consisting of a three-character identification character string indicating that the IC card is related to the present system. Further, on the back of the IC card are printed: the aforementioned patient identification code; a security code consisting of a character string of six characters; and a two-dimensional barcode symbol corresponding to the patient identification code and the security code.

Cloud Server

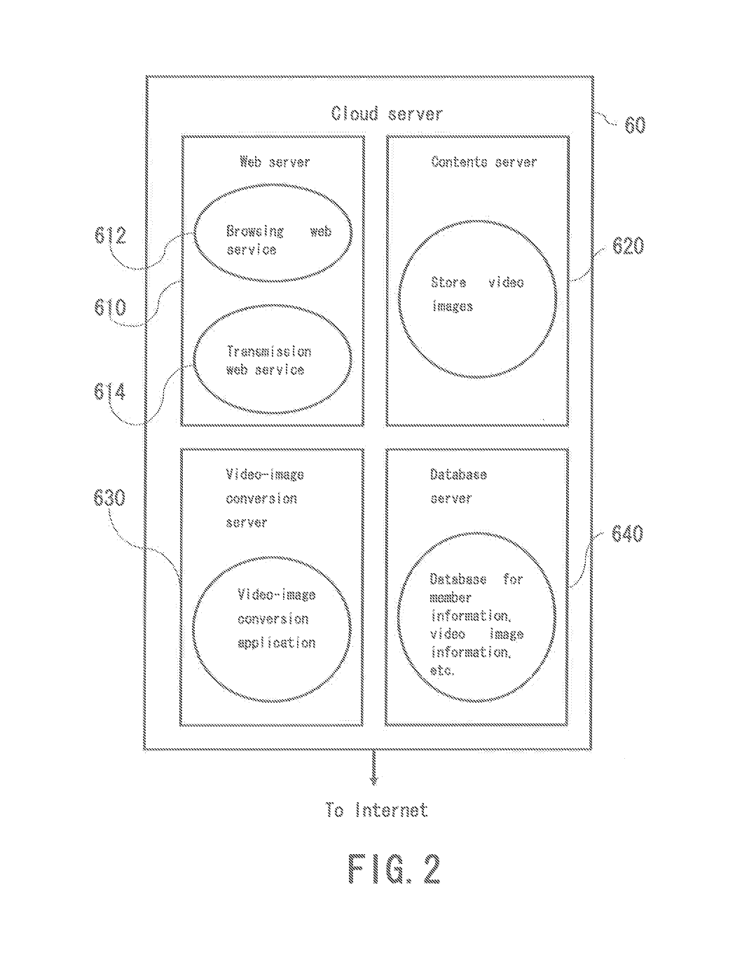

As illustrated in FIG. 2, the cloud server 60 is made up of a web server 610, a contents server 620, a video-image conversion server 630, and a database server (referred to hereinafter as DB server) 640. The web server 610 is connected to the Internet, and the other servers are connected over a LAN (local-area network) as a cloud server group. As well-known in the art, each server consists of a computer in which a CPU (central processing unit), an internal memory, input/output units, an external memory such as a HDD, and the like are combined as appropriate. Also, each server has installed thereon an OS (operating system) and software that are necessary depending on the function/role of each server.

The function/role of each server will be described below. The web server 610 performs interaction to distribute echo video images so a reception terminal 40 (described later) for browsing echo video images, and also performs interaction to receive video images from the transmission terminal unit 21. The video-image conversion server 630 converts uploaded echo video images into browsable formats compliant with the types of reception terminals 40 by using a video-image conversion application. The contents server 620 stores the converted echo video images. The DB server 640 provides a database for storing IC card information, user information, echo video image information, etc., necessary for the present system; one stored information is updated and managed as necessary.

Reception Terminal

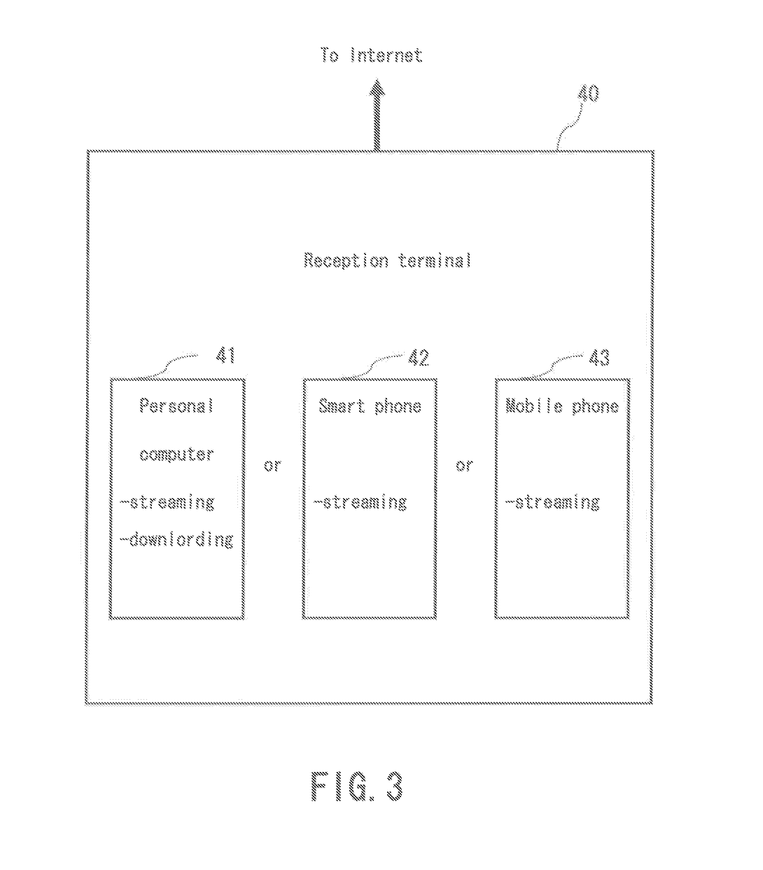

FIG. 3 illustrates an embodiment of a reception terminal. The reception terminal 40 performs user registration and browsing of echo video images from a website that is provided by a video-image browsing web service 612 of the web server 610 by making a request to the web server 610. The reception terminal 40 has a web browser installed thereon. As illustrated in FIG. 3, the reception terminal 40 may be any one of a personal computer 41, a smartphone 42, or a mobile phone 43.

Next, the processing flow of the present system will be described.

Acquiring Echo Video Image

S210

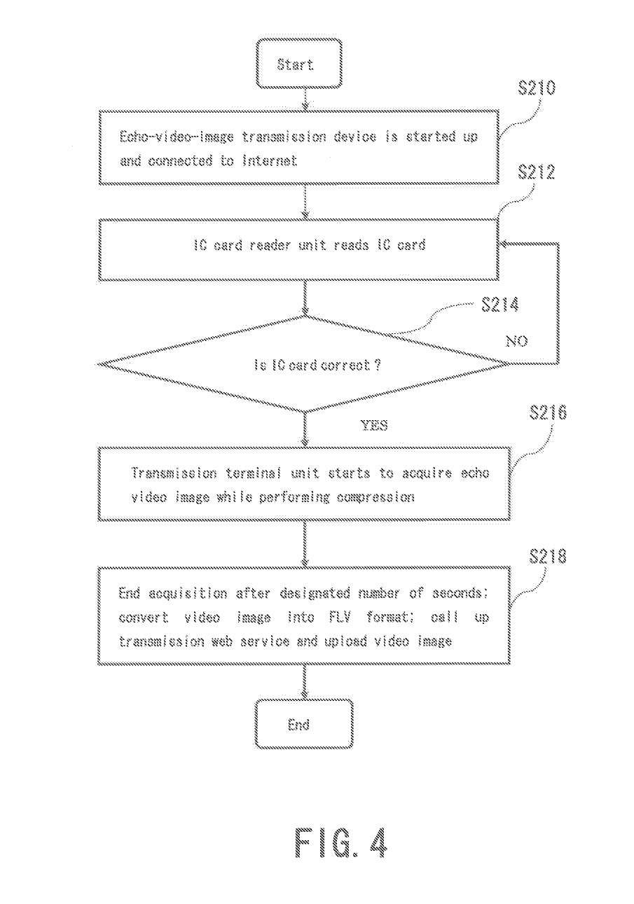

As illustrated in FIG. 4, the ultrasonic diagnostic device 10 is started up so that it can send echo video images to the laptop PC (personal computer) 21 serving as a transmission terminal unit. Also, the echo-video-image acquisition application program of the laptop PC 21, which is connected to the Internet, is started up.

S212

A predetermined IC card, which has been issued to a prenatal patient is advance, is prepared. This IC card can be read with the IC card reader 22, and information recorded thereon is read by bringing the IC card close to the IC card reader 22 which is connected to the laptop PC 21. In practice, the IC card nay be touched once lightly no the IC card reader 22.

S214

A medical institution code, which is scored in the transmission terminal unit 21 in advance, is attached to the patient identification code that has been read, and the transmission terminal unit 21 accesses the DB server 640 via the web server 610 (described later) to verify whether the medical institution code and user registration, if any, performed by the prenatal patient match the information in the DB server 640 and to verify whether the card is a correct IC card. In this way, even if the prenatal patient hands the medical institution a wrong IC card issued by another medical institution, no echo video image is acquired while mistaking the patient identification code.

S216

The reading of the IC card triggers as echo video image to be stored, as a temporary file, in a HDD (hard disk device) in the laptop PC 21 via a capture board connected to the ultrasonic diagnostic device 10, while being compressed in the AVI format, which is the standard format for video files adopted in Windows (registered trademark). The time period for acquisition can be changed by setting the echo-video-image acquisition program in advance to a fixed time period, such as 30 seconds, 60 seconds, or 90 seconds.

S218

After a lapse of the designated number of seconds, the acquisition of the echo video image is ended. The echo video image is converted into an FLV file for uploading. Then, the transmission web service process 614 (described later) of the cloud server 60 is called, and the video image is uploaded onto and stored in the contents server 620 (described later).

The aforementioned series of processes—up to this point from after the IC card is read—is performed automatically without manual operation. Thus, the only operation that the staff of the medical institution needs to perform in acquiring an echo video image is to prepare the ultrasonic diagnostic device 10 and the echo-video-image transmission device 20 in advance so that an echo video image can be acquired, and after this preparation, he/she only needs to read the IC card with the IC card reader 22 once, and the echo video image can be handed to the cloud server 60 reliably, without causing any further trouble. Moreover, no mistake is made even if information of a same type of IC card issued by a different medical institution is read.

Next, the processes of the cloud server 60 will be described. As described above, the cloud server 60 includes a plurality of servers that share various processes, and includes the web server 610, the contents server 620, the video-image conversion server 630, and the database server 640 as illustrated in FIG. 2. These servers will be described below.

Web Server 610

The web server 610 performs the following services: service for allowing the reception terminal 40 to browse echo video images; and service to the transmission terminal unit 21. More specifically, the web server 610 performs: (1) service of performing various processing, such as allowing access of the reception terminal 40 to the DB server 640 by being called up by the reception terminal 40 (“browsing web service process”); and (2) service of storing echo video image files uploaded from the transmission terminal unit 21 into the contents server 620 (“transmission web service process”).

Contents Server

The contents server 620 classifies, according to predetermined classifications, echo video image files corresponding to various types of reception terminals 40 and processed by the video-image conversion server 630 (described later), and stores the echo video image files.

DB Server

The DB server 640 stores data necessary for advancing the processes in the present system. This server mainly stores patient information data and video-image conversion information data.

Video-Image Conversion Server

The video-image conversion server 630 provides a “video-image conversion application”, and performs video-image conversion processing.

Below, the processes of each server will be described.

As described above, the web server 610 performs see browsing web service process and the transmission web service process. First, the transmission web service process will foe described.

Transmission Web Service Process

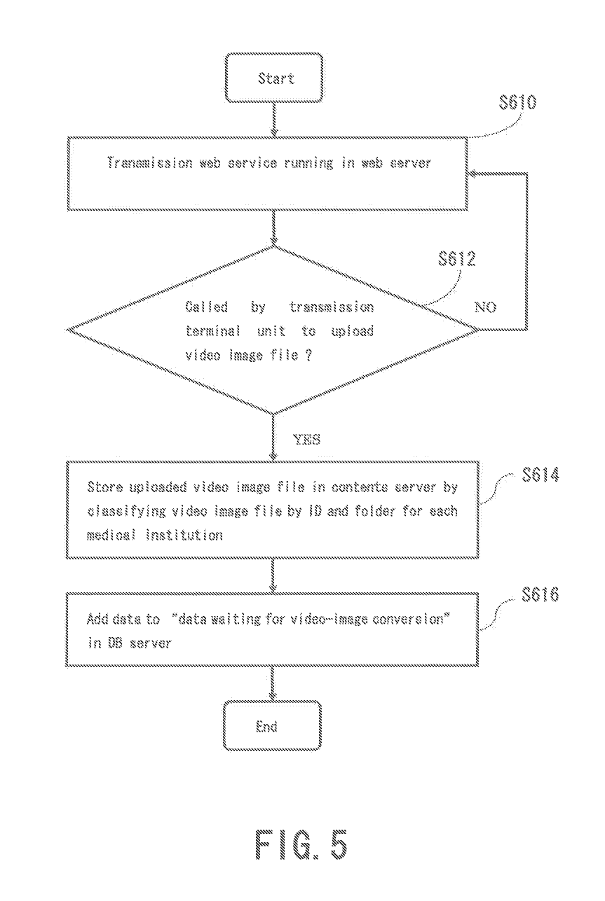

FIG. 5 illustrates the transmission web service process.

S610

The web server 610 is connected to the Internet, and the transmission web service 614 is started up so that, if there is a request from the transmission terminal unit 21, it can respond thereto.

S612

The started-up web service monitors whether if is called by the transmission terminal unit 21 to upload an echo video image file. If there is a request, the procedure moves on to the next process S614. Otherwise, the web service keeps monitoring.

S614

The requested echo video image file is uploaded onto the contents server 620. At this time, the echo video image file is classified according to the patient identification code in a folder corresponding to each medical institution.

S616

Data regarding the status information of this file is added to “data waiting for video-image conversion” in the DB server 640.

Video-Image Conversion and Storage Process

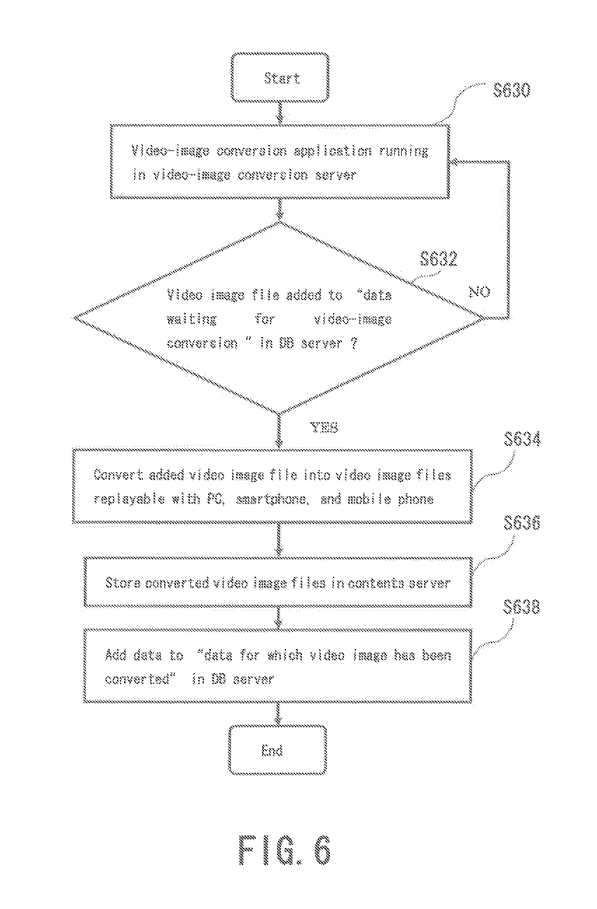

As illustrated in FIG. 6, the video-image conversion server 630 provides a “video-image conversion application” program and performs a video-image conversion process. The contents server 620 stores the echo video image file for which the video-image conversion process has been finished.

S630

The web server 610 is connected to the Internet, and the video-image conversion application is started up so that, if there is a request from the transmission terminal unit 21, it can respond thereto.

S632

The video-image conversion application monitors whether an echo video image file uploaded from the transmission terminal unit 21 has been added in the “data waiting for video-image conversion” in the DB server 640. If data has been added, the procedure moves on to the next process. Otherwise, the application keeps monitoring.

S634

The video-image conversion application converts the added echo video image files into video-image files that can be replayed by various types of reception terminals 40. Herein, “various types of reception terminals 40” ere PCs, smartphones, and mobile phones. It should be noted that, if the types of reception terminals 40 are increased, the types of conversions of video image files that can be replayed may be increased accordingly.

S636

When the conversion is finished, the converted echo video image files are stored in the contents server 620.

S638

Data is added to “data for which video image has been converted” in the DB server 640.

User Browsing Process

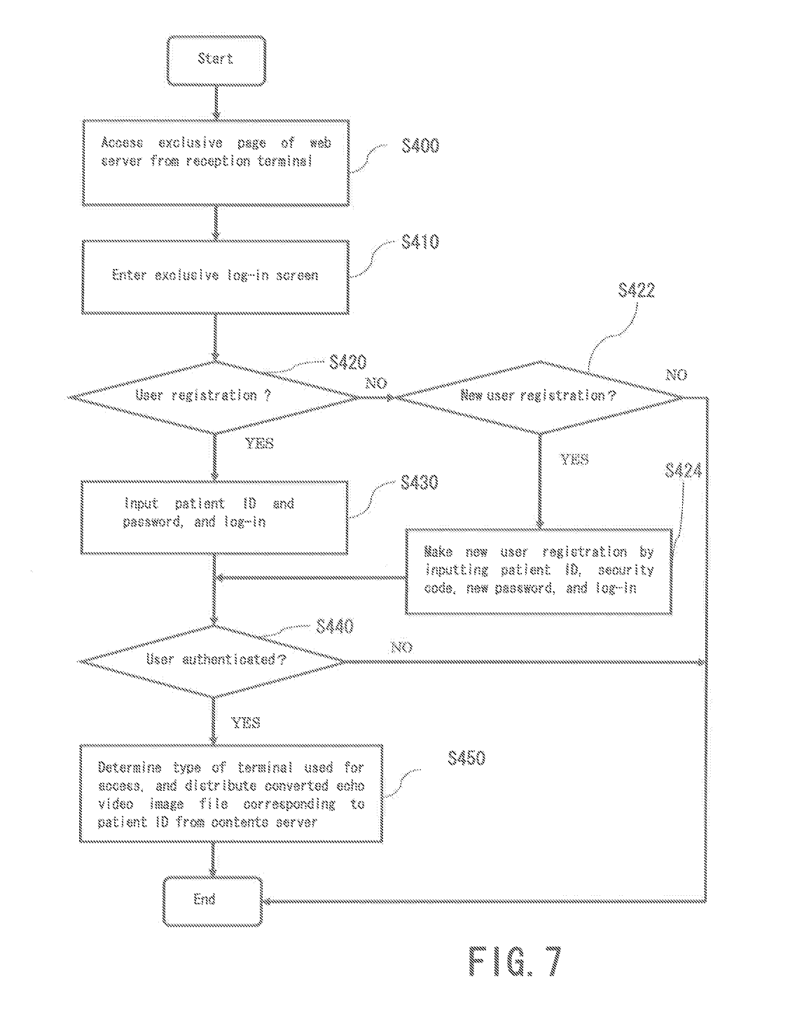

As illustrated in FIG. 7, when there is a request from a reception terminal 40 to browse an echo video image, the web server 610 is triggered to provide as browsing web service 612, and performs a user browsing process.

S400

A user sends a request to access an exclusive page (log-in screen) of the web server 610 from a reception terminal 40—i.e., a PC, a smartphone, or a mobile phone connected to the Internet and with which web browsing is possible.

S410

The reception terminal 40 displays the exclusive log-in screen sent from the web server 610. The user can enter necessary information through this screen.

S420

The user selects, from the log-in screen, whether she has already registered or not. If user registration has already been completed, the procedure moves on to the next log-in process S430. Otherwise, the procedure moves on to process S422 for new user registration.

S422

The user selects whether or not to make a new registration. If the user is to make a new user registration, the procedure moves on to the next process S424. Otherwise, the user browsing process is terminated.

S424

When the user inputs the patient identification code and the security code described on the IC card, new user registration is made and log-in processing is started, and the procedure returns to S440.

S430

Log-in processing is started when the user inputs the patient identification code and password from the log-in screen.

S440

In permitting user log-in, whether or not the user is admissible is authenticated. The authentication is performed by using the patient identification code and the password. If authenticated, the procedure moves on to the cent process S450. Otherwise, the user browsing process is terminated.

S450

The type of reception terminal used for access by the logged-in user is determined, and the converted echo video image file corresponding to the user's patient identification code and corresponding to the determined reception-terminal type (PC, smartphone, or mobile phone) is transmitted by streaming from the contents server. It should be noted that SSL-encrypted communication is used at the time of transmission.

In this way, the present system enables a user—such as a prenatal/postnatal patient or her family—to reliably browse the fetus concerned, and can minimize the trouble given to the staff of medical institutions, sued as doctors, at the time of acquiring echo video images. Further, the prenatal/postnatal patient, as well as her family and acquaintances, can browse fetal echo video images easily.

In addition, by setting up the server on a cloud (i.e., on the Internet) and not in each medical institution, the time and effort for server maintenance can be reduced significantly, and also, space securement and costs incurred by the medical institution can be reduced, thus providing an easy-to-use system.

Although an embodiment of the present invention has been described above by way of examples, the present invention is not limited to the foregoing embodiment.

1. An echo-video-image transmission device that uploads, onto a cloud server, an echo video image of a fetus of a prenatal patient that is output from an ultrasonic diagnostic device, the echo-video-image transmission device comprising
a transmission terminal unit and
an IC card reader unit, wherein,
when said IC card reader unit reads an IC card on which at least a patient identification code and a system identification code are recorded, said transmission terminal unit is triggered to start acquiring said echo video image that is output from said ultrasonic diagnostic device, and
the transmission terminal unit uploads, onto said cloud server, said echo video image that has been acquired for a predetermined period of time, together with said patient identification code and a medical institution identification code that has been stored in said transmission terminal unit in advance.

2. The echo-video-image transmission device according to claim 1, wherein,
when said IC card reader unit reads said IC card, said echo-video-image transmission device determines whether said IC card is to be used in said echo-video-image transmission device on the basis of whether said medical institution identification code matches, and
the echo-video-image transmission device starts to acquire said echo video image if said medical institution identification code matches, and does not acquire said echo video image if said medical institution identification code does not match.

3. The echo-video-image transmission device according to claim 2, wherein said echo-video-image transmission device uses user registration information stored in said cloud server in advance as a basis for determining whether said IC card is to be used in said echo-video-image transmission device.

4. A video-image distribution system comprising:
said echo-video-image transmission device according to claim 1,
said cloud server, and
a reception terminal, wherein
said cloud server stores each echo video image uploaded from said echo-video-image transmission device in association with a medical institution identification code and a patient identification code, and
when receiving a request from said reception terminal to browse said echo video image, said cloud server performs user authentication, and then distributes, by streaming or downloading, said echo video image in a format compliant with said reception terminal.

5. The video-image distribution system according to claim 4, wherein said user authentication is performed by using a password designated by a user at the time of user registration performed in advance in association with said patient identification code and a security code described on said IC card.
